(19) United States
(12) Patent Application Publication
(10) Pub. No.: US 2013/0144358 A1
(43) Pub. Date: Jun. 6, 2013

(54) Dopamine Stimulation Device

(71) Applicant: Stephen Daroe, Delray Beach, FL (US)

(72) Inventor: Stephen Daroe, Delray Beach, FL (US)

(21) Appl. No.: 13/309,900

(22) Filed: Dec. 2, 2011

(51) Int. Cl. A61N 1/36

(52) U.S. Cl. 607/45

(57) ABSTRACT

A set of eyeglasses that has a first electrical contact on the frame of the eyeglasses, and a second electrical contact on an arm of the eyeglasses. The contacts touch the skin of the wearer near the frontal cortex and behind the frontal lobe. An integral solar cell provides a small voltage output, typically 1.5 volts or less, to the contacts and stimulates the production of dopamine with a small electrical charge. Optionally, either internal or external batteries can be used to supply power in place of the solar cell.

### BACKGROUND

1. Technical Field

This invention relates in general to methods of improving brain function, and more particularly it relates to externally stimulated the production of dopamine.

2. Background of the Invention

There have been a variety of attempts to help individuals afflicted with brain function problems. A large variety of brain function conditions that affect the brain can cause severe adverse conditions, such as depression, attention deficit disorder, loss of creativity, etc. These conditions can create problems for individuals in every area of their life.

One naturally occurring neurotransmitter, dopamine, has been found to provide a variety of beneficial health effects. It can improve mood, creativity, cognition, and provide a general feeling of well-being. However, diminished levels of dopamine can have the opposite effect. It can cause diminished performance in professional and personal life for an individual. It would be desirable to have a method of restoring dopamine to desirable levels such that individuals could perform at a higher level.

The pharmaceutical industry has developed a variety of pharmaceuticals that can help individuals having certain conditions, such as low dopamine levels. One method of increasing the levels of dopamine in an individual's body has been through the use of pharmaceuticals. While pharmaceuticals can produce increases in dopamine levels, they also have drawbacks. Namely, they can be expensive for the user, they require users to maintain a schedule of dosage that may be inconvenient, and the pharmaceuticals may conflict with other pharmaceuticals that a user is taking which may result in adverse side effects. It would be desirable to have a method of increasing dopamine levels in an individual's body without requiring ingestion of pharmaceuticals.

It has been found that stimulation of the brain by electrical probes results in increased production of dopamine when applied properly. While this method overcomes the need for pharmaceuticals, the requirement for special equipment also makes this method impractical. It would be desirable to have a method of stimulating that produces dopamine via external electrical stimulation without the requirement for cumbersome and expensive equipment.

While the prior art has provided basic treatments for low dopamine levels, it has failed to provide an inexpensive portable system that can inexpensively and conveniently provide increased production of dopamine levels through electrical stimulation.

### SUMMARY OF THE INVENTION

This invention provides a set of eyeglasses that has an electrical contact on the frame and a second electrical contact on an arm of the eyeglasses. The contacts touch the skin of the wearer. An integral solar cell provides a small voltage output, typically 1.5 volts or less, to the contacts and stimulates the production of dopamine with a small electrical charge.

### BRIEF DESCRIPTION OF THE DRAWINGS

FIG. 1 is a front view of a preferred embodiment of an eyeglass frame that incorporates the invention.

FIG. 2 is a top view of a preferred embodiment of an eyeglass frame that incorporates the invention.

FIG. 3 is a side view of a preferred embodiment of an eyeglass frame that incorporates the invention and illustrates a tethered solar cell.

FIG. 4 is a side view of a preferred embodiment of an eyeglass frame that incorporates the invention and illustrates an integrated solar cell.

FIG. 5 is a side view of a preferred embodiment of an eyeglass frame that incorporates the invention and illustrates a tethered battery pack.

FIG. 6 is a side view of a preferred embodiment of an eyeglass frame that incorporates the invention and illustrates an integrated battery.

### DETAILED DESCRIPTION OF THE PREFERRED EMBODIMENT

Prior to a discussion of the figures, an overview of the invention will be presented. The invention is a portable electronic device that stimulates the production of dopamine the administration of very low voltage (1.5 V or less) the frontal lobe of an individual. In the preferred embodiment, the device is incorporated into a conventional eyeglass frame. The device has two contacts that touch the skin of the user. The first contact uses a wire that extends from the bridge of the eyeglasses to the skin of the user were the frontal cortex. The second contact extends from an arm of the eyeglasses and touches the skin of the user near the distal end of the eyeglass arm near the user's ear and behind the frontal lobe. In practice, contacts can be held in place on the user's skin via EKG tape or any other suitable method.

The application of a low voltage in this manner stimulates neurotransmitters and improves brain function and cognizance by increasing the natural production of dopamine within the user's brain.

The device has a power source that supplies the low voltage to the contacts. In a preferred embodiment, a solar cell is used which can be attached to the user in any convenient manner. It can be worn as a necklace, as a belt buckle, placed in the user's pocket, etc. This solar cell allows the user to wear the device continuously without concern as the loss of power when outdoors or in bright light locations.

An alternative embodiment of the invention uses a solar cell that is integrated into the eyeglass frame such that the external solar cell discussed in regard to the prior embodiment is not needed. However, the solar cells used in both embodiments requires substantial amounts of light to generate the required electrical charge. As a result, the device would not work in dark indoor locations, or outside at night.

Another embodiment of the invention uses an extra battery pack that is tethered to the eyeglass frame. This requires the inconvenience of periodically replacing batteries, but it also provides the advantage of being usable in any lighting situation.

Yet another embodiment of this invention uses a battery that is integrated into the eyeglass frame. This embodiment provides an extra convenience in that there are no external components attached to the eyeglass frames.

Having discussed the invention in general, we turn now to a detailed discussion of the drawings.

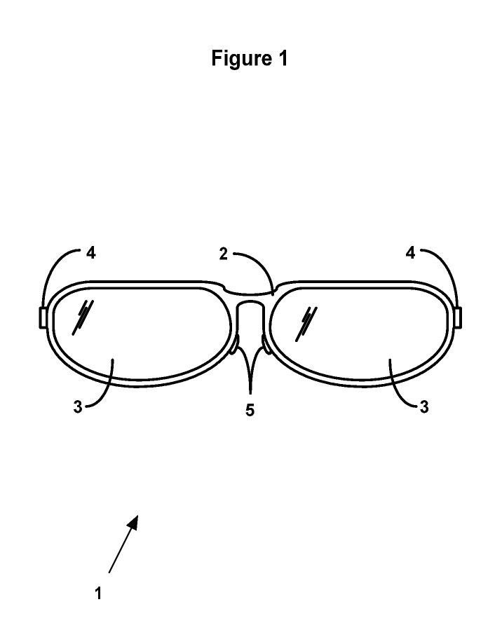

Regarding FIG. 1 is a front view of a preferred embodiment of a set of eyeglasses 1 that incorporates the invention. This figure illustrates the frame 2, the lenses 3, the arms 4, in the nose cushions 5. In this view, the functional features of the invention are not visible. An observer would merely see a conventional set of eyeglasses 1.

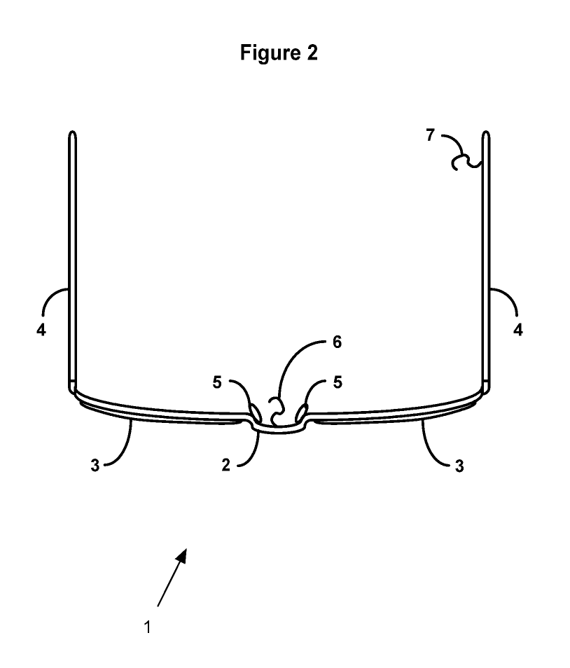

FIG. 2 is a top view of the preferred embodiment of the set of eyeglasses 1 illustrated in FIG. 1. Also shown in this figure are the front contact 6, and the rear contact 7. During normal use, the front contact 6 would be held against the skin of the user by EKG tape or other suitable methods to ensure a good electrical connection between the front contact 6 and the use of skin. Likewise, the rear contact 7 would also be held against the skin of the user in like manner. The voltage difference between front contact 6 and rear contact 7 would provide a stimulating current that would improve brain function by increasing dopamine production by the brain.

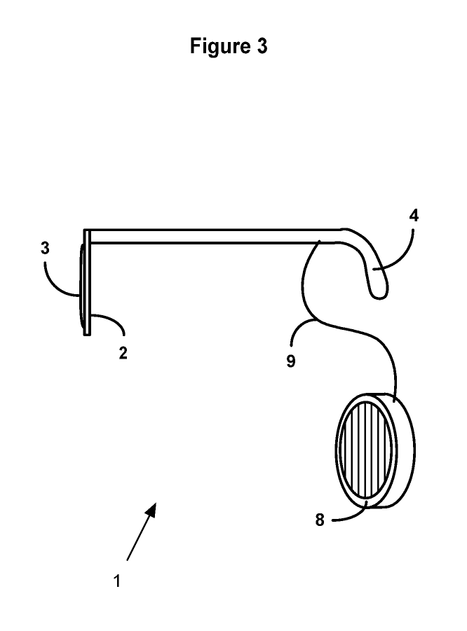

FIG. 3 is a side view of a preferred embodiment of the set of eyeglasses 1 discussed in regard to FIGS. 1-2. This figure illustrates a tethered solar cell 8 that is electrically attached to the set of eyeglasses 1 via cable 9. This configuration provides a constant source of power so long as the user is in a brightly lit area, or the user is in daylight. The solar cell 8 can be worn attached to the user in any number of convenient locations. It can be structured as a belt buckle, a jewelry item, a badge, or any other aesthetically pleasing design that would disguise its functionality.

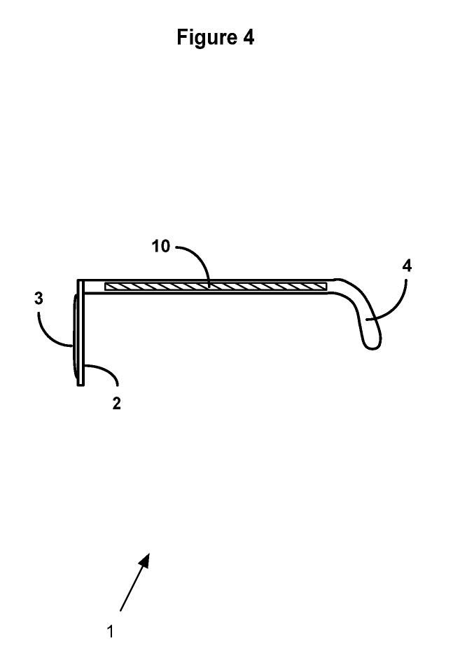

FIG. 4 is a side view of a preferred embodiment of the set of eyeglasses 1 that incorporates the previously discussed features of the invention and further includes a solar cell that is integrated into the frame of the eyeglasses. The advantage of this embodiment is that it eliminates any external equipment, such as a solar cell, and makes it easier to carry the device.

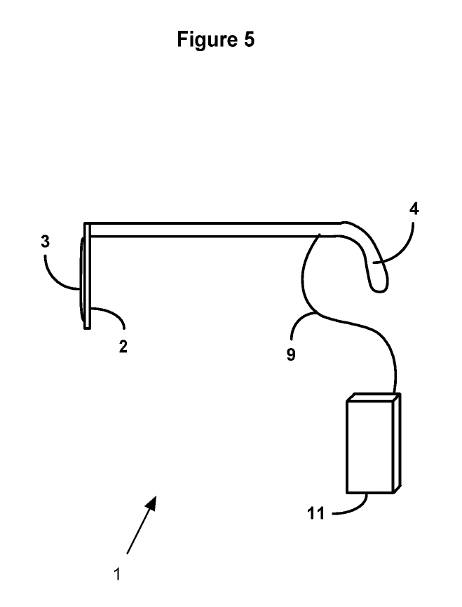

FIG. 5 is a side view of a preferred embodiment of the set of eyeglasses 1 that incorporates the previously discussed features of the invention and further illustrates a battery pack 11 that is tethered to the arm 4 via cable 9. This embodiment provides the same convenience offered by the previous embodiments, but also allows the device to be used in any lighting environment.

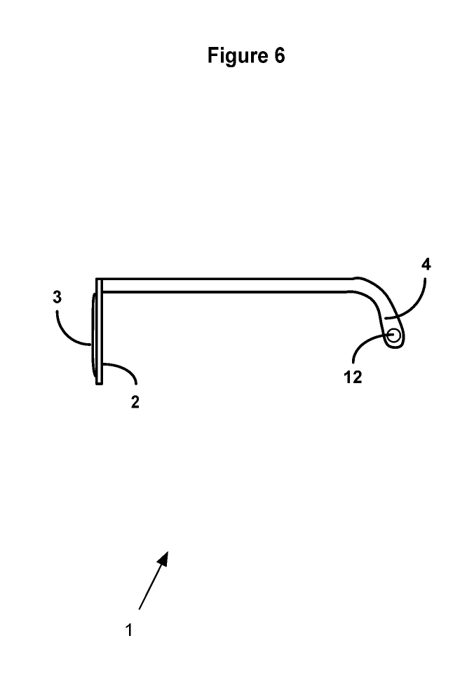

FIG. 6 is a side view of a preferred embodiment of the set of eyeglasses 1 that incorporates the previously discussed features of the invention and further illustrates a battery 12 that is integrated into arm 4. This embodiment provides the same convenience offered by the previous embodiments, but also eliminates any external equipment and allows the device to be used in any lighting environment.

While specific embodiments have been discussed to illustrate the invention, it will be understood by those skilled in the art that variations in the embodiments can be made without departing from the spirit of the invention. The types of solar cells can vary, the types of battery power can vary, the location of the solar cells can be external to the set of eyeglasses 1 or integrated with it. Therefore, the invention shall be limited solely to the scope of the claims.

I Claim:

1. A brain function stimulation device, comprising:
a set of eyeglasses having a frame, and arms;
at least one front contact extending from the frame a sufficient distance to contact the surface of a user's skin near the frontal cortex;
at least one rear contact extending from the arm a sufficient distance to contact the surface of the user's skin behind the frontal lobe;
a power source that provides a low voltage approximately 1.5 V or less to the contacts such that a low-level current passes through the brain causing increased production of dopamine;
whereby the dopamine produced by the brain improved overall brain function.

2. A device, as in claim 1, wherein:
the contacts are secured to the skin surface of the user.

3. A device, as in claim 2, wherein:
the contacts are secured to the skin surface of the user with EKG tape.

4. A device, as in claim 1, wherein:
the power source is a solar cell externally connected to the set of eyeglasses and electrically connected to the contacts.

5. A device, as in claim 4, wherein:
the contacts are secured to the skin surface of the user.

6. A device, as in claim 5, wherein:
the contacts are secured to the skin surface of the user with EKG tape.

7. A device, as in claim 1, wherein:
the power source is a solar cell integrated into the body of the set of eyeglasses and electrically connected to the contacts.

8. A device, as in claim 7, wherein:
the contacts are secured to the skin surface of the user.

9. A device, as in claim 8, wherein:
the contacts are secured to the skin surface of the user with EKG tape.

10. A device, as in claim 1, wherein:
the power source is a battery pack externally connected to the set of eyeglasses and electrically connected to the contacts.

11. A device, as in claim 10, wherein:
the contacts are secured to the skin surface of the user.

12. A device, as in claim 11, wherein:
the contacts are secured to the skin surface of the user with EKG tape.

13. A device, as in claim 1, wherein:
the power source is a battery that is integrated into the set of eyeglasses and electrically connected to the contacts.

14. A device, as in claim 13, wherein:
the contacts are secured to the skin surface of the user.

15. A device, as in claim 14, wherein:
the contacts are secured to the skin surface of the user with EKG tape.

16. A method of improving brain function by stimulating dopamine production, including the steps of:
attaching a first contact to a user's skin near the frontal cortex, and attaching a second contact to a user's skin behind the frontal lobe;
increasing the production of dopamine in a user's brain by applying a low voltage to the first and second contacts such that a small voltage is applied to the user's brain.

17. A method, as in claim 16, including the additional step of:
using 1.5 V or less as the low voltage.

18. A method, as in claim 17, including the additional step of:
integrating the contacts and to a set of eyeglass frames.

19. A method, as in claim 18, including the additional step of:
using a solar cell to supply the low voltage.

20. A method, as in claim 18, including the additional step of:
using a battery to supply the low-voltage.
